(19) United States
(12) Patent Application Publication
(10) Pub. No.: US 2023/0248309 A1
(43) Pub. Date: Aug. 10, 2023

(54) COMPUTER-IMPLEMENTED METHOD

(71) Applicant: ROCKLEY PHOTONICS LIMITED, Altrincham (GB)

(72) Inventors: Jennifer Lynn CORSO, Peoria, AZ (US); Mathew PAUL, Irvine, CA (US)

(21) Appl. No.: 18/055,375

(22) Filed: Nov. 14, 2022

Related U.S. Application Data
(60) Provisional application No. 63/279,633, filed on Nov. 15, 2021.

(51) Int. Cl. A61B 5/00; G16H 40/67; G16H 50/30

(52) U.S. Cl. CPC A61B 5/4875; A61B 5/0082; A61B 5/7221; A61B 5/6801; G16H 40/67; G16H 50/30

(57) ABSTRACT

A computer-implemented method for determining a hydration status of a user, the computer-implemented method. The computer-implemented method comprises acquiring, from sensor on a wearable device worn by a user, data including bodily parameter data related to the user. The computer-implemented method further comprises applying a model to the bodily parameter data to obtain hydration information related to the user. The model derives, from the hydration information, a hydration rank indicative of a hydration status of the user. The hydration rank is a given grade on a hydration rank scale.

### CROSS-REFERENCE TO RELATED APPLICATION(S)

The present application claims priority to and the benefit of U.S. Provisional Application No. 63/279,633, filed Nov. 15, 2021, entitled “COMPUTER-IMPLEMENTED METHOD FOR A WEARABLE DEVICE”, the entire content of which is incorporated herein by reference.

### FIELD

One or more aspects of embodiments according to the present invention relate to a computer-implemented method for determining a hydration status of a user.

### BACKGROUND

Hydration status may have a significant impact in several areas, including a person's mood, physical and mental performance, kidney function, and skin condition. Understanding body hydration and when it might not be balanced properly can be extremely valuable for managing personal health and well-being.

However, the model surrounding hydration is exceptionally complex. Fluid balance is sustained through the coordination of many substances and mechanisms working in concert, with a heavy reliance upon neuroendocrine responses and healthy renal function. Modifying any one part of the fluid environment through dehydration or overhydration may elicit profound effects on hemodynamics and overall function.

As it stands, there exists no “gold standard measurement” for assessing hydration status, and therefore, no simplified index. To determine a person's level of hydration, various costly and time-consuming laboratory tests are employed to assess the osmolarity and electrolyte concentrations in physiological fluids such as urine and blood. Laboratory tests may include urine osmolality (UO), urine specific gravity (USG), serum osmolality (SO), fluid gain and fluid loss.

Fluid imbalances may lead to serious physical complications and present in a variety of common scenarios where laboratory testing is not available, timely, nor practical.

### SUMMARY

Accordingly, embodiments of the present invention provide a computer-implemented method for determining a hydration status of a user, the computer-implemented method comprising acquiring, from sensor on a wearable device worn by a user, data including bodily parameter data (for example in the form of an absorption spectrum) related to the user, and applying a model to the bodily parameter data to obtain hydration information related to the user, wherein the model derives, from the hydration information, a hydration rank indicative of a hydration status of the user, wherein the hydration rank is a given grade on a hydration rank scale.

Thus, an indication of the hydration status of a user can be derived from wearable device-acquired data. In this way, an indication of the hydration status of the user may be provided during a user's normal routine. Advantageously then, an indication of the hydration status of the user may be provided in a more accessible way than can be provided by laboratory tests.

Furthermore, daily water intake by the user may be improved and the risk of dehydration decreased. Cognitive and physical performance may be improved as a result. Use of the methods and devices described herein may therefore contribute to: decreased rates of hospital admission due to dehydration and/or overhydration; potential decreases in hospital costs such as IV fluids, laboratory tests, staffing and related fees; improvements in symptoms related with anxiety, depression and/or PTSD; decreased risk of thermal injury such as accidental hyperthermia; decreased risk of kidney stones; improved dialysis and diuresis treatments; decreased risk of hypovolemia or volume depletion; decreased risk of hypotension; optimized dietary fluid restriction or similar; optimized fluid administration and/or improved survival rates during shock and trauma.

Optional features of the computer-implemented method will now be described. The computer-implemented method may have any one, or any combination insofar as they are compatible, of the following features.

The computer-implemented method may be carried out on the wearable device, or on an external device. The external device may be a mobile device such as a mobile phone. The wearable device may be any device worn on the body, for example a watch, a ring, a necklace, bracelet, an ear bud, a skin contact patch, a glasses frame, or a strap worn around the wrist, the arm, the leg, or the torso.

The hydration status of the user may be a clinical categorization, or a clinical hydration status. The hydration status of the user may be hypovolemia, euvolemia, or hypervolemia. The hydration status of the user may be severe hypovolemia, moderate hypovolemia, mild hypovolemia, euvolemia, mild hypervolemia, moderate hypervolemia or severe hypervolemia. A hydration status of hypovolemia may include hydration statuses of mild hypovolemia, moderate hypovolemia and severe hypovolemia. A hydration status of hypervolemia may include hydration statuses of mild hypervolemia, moderate hypervolemia and severe hypervolemia.

Hypovolemia may be referred to as dehydration. Hypervolemia may be referred to as overhydration. Euvolemia may be referred to as normal hydration. Mild hypovolemia, euvolemia and mild hypervolemia may be referred to as normal hydration.

The bodily parameter data may be an optical spectrum. The bodily parameter data may be an absorption spectrum. The bodily parameter data may be a body tissue absorption spectrum. The absorption may be in the water band.

The hydration information may be a quantitative value associated with the bodily parameter data. The hydration information may be a quantitative value associated with the absorption spectrum. For example, the hydration information may be a position of a peak of the absorption spectrum, for example a wavelength shift of a peak of the absorption spectrum, a height of a peak of the absorption spectrum, or a width of a peak of the absorption spectrum.

Each hydration rank on the hydration rank scale may map onto a respective output of a standard clinical point of care test. Each hydration rank on the hydration rank scale may map onto a respective range of outputs of a standard clinical point of care test.

In this way, a hydration rank may be derived which provides a clinically relevant assessment of hydration status.

The standard clinical point of care test may be a body mass measurement, a test performed on a urine sample, or a test performed on a blood sample. The standard clinical point of care test may be a test of urine osmolality (UO), urine specific gravity (USG), fluid gain or weight gain, fluid loss or weight loss, or serum osmolality (SO). The standard clinical point of care test may be a test of temperature. The standard clinical point of care test may be a test of heart rate. The standard clinical point of care test may be a test of urine color, a test of urine volume, a test of skin turgor, a test of jugular venous distention, or an ultrasound test. The standard clinical point of care test may define ranges of outputs of the standard clinical point of care test, wherein each of the ranges corresponds to a respective clinical hydration status.

An output of a UO test may have units of mOsm/kg. An output of a UO test which is <80 may indicate overhydration. An output of a UO test which is <500 may indicate overhydration. An output of a UO test which is >80 to <500 may indicate overhydration, with values below 80 mOsm/kg considered critical. An output of a UO test which is between approximately 500 and 700 may indicate normal hydration. An output of a UO test which is between approximately 700 and >1200 may indicate dehydration.

An output of a USG test which is within the range 1.01 to <1.005 may indicate overhydration. An output of a USG test which is within the range 1.005 to <1.020 may indicate normal hydration. An output of a USG test which is within the range 1.020 to 1.040 may indicate dehydration.

An output of an SO test may have units of mOsm/kg. An output of an SO test which is <265 may indicate overhydration. An output of an SO test which is <285 may indicate overhydration. An output of an SO test which is >265 to <285 may indicate overhydration. An output of an SO test which is within the range 285 to <295 may indicate normal hydration. An output of an SO test which is within the range 295 to 320 may indicate dehydration.

An output of a weight loss test may have units of % of body mass. An output of a weight loss test which is weight gain indicates overhydration. An output of a weight loss test which is within the range 0% to <2% may indicate normal hydration. An output of a weight loss test which is within the range 2% to <6% may indicate mild dehydration. An output of a weight loss test which is within the range 6% to <10% may indicate moderate dehydration. An output of a weight loss test which is greater than or equal to 10% may indicate severe dehydration.

Each hydration rank on the hydration rank scale may map onto respective outputs of a plurality of standard clinical point of care tests. For example, a hydration rank may map onto an output of a urine osmolality test and an output of a urine specific gravity test. Each hydration rank on the hydration rank scale may map onto respective ranges of outputs of a plurality of standard clinical point of care tests.

The plurality of standard clinical point of care tests may include two or more of a test of urine osmolality (UO), a test urine specific gravity (USG), a test fluid gain or weight gain, a test fluid loss or weight loss, a test serum osmolality (SO), a test temperature, a test heart rate, a test urine color, a test urine volume, a test skin turgor, a test jugular venous distention, and an ultrasound test.

In this way, each hydration rank on the hydration rank scale may relate to a combination of standard clinical point of care tests. Thus, a more accurate indication of a user hydration status may be provided, because the accuracy of the hydration status indicated by the hydration rank is not limited by the inaccuracies of a single standard point of care test.

The computer-implemented method may comprise outputting an output to the user. The output may be output to the user in real time. The output may be output to the user at predetermined time points, for example the output may be output to the user every 30 minutes, every hour, every 6 hours, every 12 hours or every 24 hours. The output may be output to the user upon receiving a user input signal. Outputting the output to the user may mean displaying the output on the wearable device. Outputting the output to the user may mean displaying the output on an external device, which may be a mobile device such as a mobile phone.

In this way, information may be provided to the user in a more accessible way than can be provided by laboratory tests.

The output may include the hydration rank.

Further, the turnaround time of results in clinical scenarios may be shortened.

The computer-implemented method may comprise deriving a time-averaged hydration rank.

The output may include the time-averaged hydration rank.

In this way, a more accurate indication of the hydration status of the user may be provided.

The computer-implemented method may continuously acquire data. The computer-implemented method may continuously derive the hydration rank. The computer-implemented method may acquire data at pre-determined time points, for example the data may be acquired every 30 minutes, every hour, every 6 hours, every 12 hours or every 24 hours. The computer-implemented method may acquire data upon receiving a user input signal. The computer-implemented method may derive the hydration rank at predetermined time points, for example the hydration rank may be derived every hour, every 6 hours, every 12 hours or every 24 hours. The computer-implemented method may derive the hydration rank upon receiving a user input signal.

The hydration rank may include a hydration index. The hydration index may be a given value on a hydration index scale.

A value may mean a numerical value.

In this way, a hydration index scale may be derived which provides a quantitative assessment of hydration status.

Each hydration index on the hydration index scale may map onto a respective output of the standard clinical point of care test. Each hydration index on the hydration index scale may map onto a respective range of outputs of a standard clinical point of care test.

In this way, a hydration index may be derived which provides a clinically relevant quantitative assessment of hydration status.

In one or more embodiments, the presentation of hydration status to the user may be designed in a way that credibly assists the user. For example, a display may include a color output, where the color is associated with hydration status. Alternatively, or additionally, visual indicators such as arrows may be presented to the user.

The standard clinical point of care test may be a body mass measurement, a test performed on a urine sample, or a test performed on a blood sample. The standard clinical point of care test may be a test of urine osmolality, urine specific gravity, fluid gain or weight gain, fluid loss or weight loss, or serum osmolality. The standard clinical point of care test may be a test of temperature. The standard clinical point of care test may be a test of heart rate. The standard clinical point of care test may be a test of urine color, a test of urine volume, a test of skin turgor, a test of jugular venous distention, or an ultrasound test.

Each hydration index of the hydration index scale may map onto respective outputs of a plurality of standard clinical point of care tests. For example, a hydration index may map onto an output of a urine osmolality test and an output of a urine specific gravity test. Each hydration index of the hydration index scale may map onto respective ranges of outputs of a plurality of standard clinical point of care tests.

In this way, each hydration index on the hydration index scale may relate to a combination of standard clinical point of care tests. Thus, a more accurate indication of a user hydration status may be provided, because the accuracy of the hydration status indicated by the hydration index is not limited by the inaccuracies of a single standard point of care test.

The hydration index scale may be sub-divided into a plurality of sub-ranges of hydration index values, each of the plurality of sub-ranges corresponding to a different clinical hydration status of the user. The computer-implemented method may comprise determining which sub-range of the plurality of sub-ranges the hydration index value falls within. The computer-implemented method may comprise determining, based upon the determined sub-range, the clinical hydration status of the user. The computer-implemented method may comprise outputting the clinical hydration status of the user, in addition or alternatively, the computer-implemented method may comprise outputting the hydration index.

Each index value on the hydration index scale may be indicative of a clinically-determined hydration status. A clinical hydration status of a user may be determined.

The hydration index scale may consist of hydration index values which are integers. The hydration index scale may comprise any number of hydration indices, for example the hydration index scale may comprise 5, 10, 15 or 20 hydration indices.

The hydration index scale may vary depending upon the spectral resolution of the sensor. The hydration index scale may depend upon the use case. The use case may be selectable by the user.

For example, a hydration index scale for a use case focused on dehydration may consist of negative hydration indices.

The hydration index scale may run from a lower value, e.g., −5, to an upper value, e.g. +5. A hydration index scale which runs from the lower value to the upper value (e.g. −5 to +5) may be applicable to critical care users. Alternatively, it may be applicable to healthy, high risk users such as athletes.

A given hydration index value between the upper and lower value (e.g. a hydration index value of 0) may indicate euvolemia. A sub-range of hydration index values (e.g. a first sub-range including negative hydration index values of e.g. −1, −2, −3, −4 and −5) may indicate dehydration. A smaller sub-range within the first sub-range (e.g. a sub-range including hydration index values of −1 and −2) may indicate mild dehydration. An alternative sub-range also within the first sub-range, but at greater negative values (e.g. a sub-range including hydration index values of −3 and −4) may indicate moderate dehydration. A hydration index value at or near the lower value (e.g. a hydration index of −5) may indicate severe dehydration. A second sub-range including, for example, positive hydration index values of, e.g. +1, +2, +3, +4 and +5 may indicate overhydration. A sub-range within the second sub-range (e.g. including hydration index values of +1 and +2) may indicate mild overhydration. A further sub-range including, for example, hydration index values of greater magnitude (e.g. hydration index values of +3 and +4) may indicate moderate overhydration. A hydration index value at or near the greatest hydration index value (e.g. of +5) may indicate severe overhydration.

A hydration index value, e.g. the lower value of the range (e.g. of −5) may correspond to USG values of over 1.030. A sub-range including hydration index values (e.g. of −3 and −4) may correspond to USG values of between 1.020 and 1.030. A sub-range (e.g. including hydration index values of −1 and −2) may correspond to USG values of approximately 1.020. A sub-range including hydration index values (e.g. of −1, −2, −3, −4 and −5) may correspond to USG values of between 1.020 and 1.040. A sub-range including hydration index values (e.g. of −2, −1, 0, 1 and 2) may correspond to UDG values of between 1.005 and 1.020. A central hydration index value (e.g. of 0) may correspond to USG values of approximately 1.010. A sub-range including hydration index values (e.g. of +1 and +2) may correspond to a USG value of approximately 1.005. A sub-range including hydration index values (e.g. of +1, +2, +3, and +4) may correspond to USG values of between 1.002 and 1.005. A hydration index value (e.g. of +5) may correspond to USG values of less than 1.002.

A hydration index value at the lower end of the range (e.g. of −5) may correspond to UO values of greater than and including 1200. A first sub-range including hydration index values towards the lower half of the range (e.g. of −1, −2, −3 and −4) may correspond to UO values of between 700 and 1200. A smaller sub-range within the first sub-range including hydration index values (e.g. of −1 and −2) may correspond to UO values of between 700 and 850. A central hydration index value (e.g. a hydration value of 0) may correspond to a UO value of approximately 500. A central sub-range including hydration index values (e.g. of −1, 0 and 1) may correspond to UO values of between 500 and 700. A sub-range including hydration index values at the upper half of the range (e.g. of +1, +2, +3, +4 and +5) may correspond to UO values of <500. A hydration index value at or near the upper end of the range (e.g. a hydration index value of +5) may correspond to UO values below 80 or below 50.

A hydration index value at or near the lower end of the range (e.g. a hydration index value of −5) may correspond to SO values of greater than 320. A sub-range including hydration index values (e.g. of −1 and −2) may correspond to SO values between 295 and 300. A sub-range including hydration index values at the lower half of the range (e.g of −1, −2, −3, −4 and −5) may correspond to SO values greater than and including 295. A central hydration index value (e.g. a hydration index value of 0) may correspond to SO values between 280 and 295. A sub-range including hydration index values at the upper half of the range (e.g. hydration index values of +1, +2, +3, +4 and +5) may correspond to SO values less than 280. A hydration index value at or near the upper end of the range (e.g. of +5 may correspond to SO values less than 265).

A hydration index value at or near the lower end of the range (e.g. of −5) may correspond to fluid loss values of greater than and equal to 10%. A sub-range including greater negative hydration index values (e.g. of −3 and −4) may correspond to fluid loss values of approximately between 6% and 10%. A further sub-range including smaller negative hydration index values (e.g. of −1 and −2) may correspond to fluid loss values of approximately between 2% and 6%. A central hydration index value (e.g. a hydration index value of 0) may correspond to fluid loss values of approximately between 0% and 2%. A sub-range including hydration index values of +1, +2, +3, +4 and +5 may correspond to SO values which indicate weight gain.

A central sub-range (e.g. including hydration index values at either side of and including the mid-point of the range e.g. of −1, 0 and +1) may indicate euvolemia. A sub-range including negative hydration index values of greater magnitude (e.g. hydration index values of −2, −3, −4 and −5) may indicate dehydration. A sub-range including negative hydration index values of a lower magnitude (e.g. −2 and −3) may indicate mild dehydration. A hydration index value of medium magnitude (e.g. −4) may indicate moderate dehydration. A hydration index value at the lower end of the range (i.e. a negative value of greatest magnitude, e.g. of −5) may indicate severe dehydration. A sub-range including hydration index values (e.g. of positive values such as +2, +3, +4 and +5) may indicate overhydration. A hydration index value of greatest magnitude at the upper end of the range (e.g. a hydration index value of +5) may indicate water intoxication.

A hydration index value at or near the lower end of the range (e.g. a hydration index value of −5) may correspond to USG values of over 1.030. A sub-range including hydration index values in the lower half of the range (e.g. negative hydration index values with lower magnitudes of e.g. −2, −3 and −4) may correspond to USG values of approximately 1.020. A sub-range including hydration index value of medium magnitude (e.g. −4) may correspond to USG values of between 1.020 and 1.030. A sub-range including central hydration index values (e.g. values either side of and including the mid-point of the range, e.g. hydration index values of −1, 0, 1) may correspond to USG values of approximately 1.010. A sub-range including hydration index values e.g. of −4, −3, −2, −1, 0, 1, 2, 3, 4 may correspond to USG values of between 1.005 and 1.020. A sub-range including hydration index values of values in the upper half of the range but not including the upper end of the range (e.g. +2, +3, +4) may correspond to a USG value of approximately 1.005. A hydration index value at the upper end of the range (e.g. of +5) may correspond to USG values of less than 1.002.

A hydration index value at the lower end of the range (e.g. of −5) may correspond to UO values of greater than and including 1200. A sub-range at the lower half of the range but not including the lowest end of the range (e.g. including hydration index values of −2, −3 and −4) may correspond to UO values of between 700 and 1200. A sub-range including hydration index values of e.g. −2 and −3 may correspond to UO values of between 700 and 850. A hydration index value at the mid-point of the range (e.g. a hydration index value of 0) may correspond to a UO value of approximately 500. A sub-range including central hydration index values (e.g. values either side of and including the mid-point of the range e.g. values of −1, 0 and 1) may correspond to UO values of between 500 and 700, or between 500 and 650. A sub-range including hydration index values at the upper half of the range (e.g. of +2, +3, +4 and +5) may correspond to UO values of <500. A sub-range including hydration index values at the upper half of the range, but not including the upper end of the range (e.g. values of +2, +3, and +4) may correspond to UO values of between 80 and 500. A hydration index value at or near the upper end of the range (e.g. of +5) may correspond to UO values below 80 or below 50.

A hydration index value at or near the lower end of the range (e.g. a value of −5) may correspond to SO values of greater than 320. A sub-range including hydration index values at the lower end of the range but not including the lowest end value of the range (e.g. values of −2, −3 and −4) may correspond to SO values between 295 and 300. A sub-range including hydration index values e.g. of −2 and −3 may correspond to SO values greater than and including 295 and less than 300. A sub-range including central hydration index values either side of and including the mid-point of the range (e.g. of −1, 0 and 1) may correspond to SO values between 280 and 295. A sub-range of values at the upper end of the range, e.g. including hydration index values of +2, +3, +4 and +5 may correspond to SO values less than 280. A hydration index value at the upper end value (e.g. of +5) may correspond to SO values less than 265.

A hydration index value at the lower end of the range (e.g. of −5) may correspond to fluid loss values of greater than and equal to 10%. A sub-range including a hydration index value in the lower half of the range but not at the lowest endpoint of the range (e.g. −4) may correspond to fluid loss values of approximately between 6% and 10%. A sub-range including hydration index values of e.g. −2 and −3 may correspond to fluid loss values of approximately between 2% and 6%. A sub-range including central hydration index values including values either side of and including the mid-point of the range (e.g. of −1, 0 and 1) may correspond to fluid loss values of approximately between 0% and 2%. A sub-range including hydration index values at the upper half of the range (e.g. of +2, +3, +4 and +5) may correspond to SO values which indicate weight gain.

The hydration index scale may run from 0 to −9. A hydration index scale which runs from 0 to −9 may be applicable to healthy and vulnerable users.

A sub-range including hydration index values at the upper end of the range (e.g. of 0, −1 and −2) may indicate euvolemia. A sub-range including hydration index values at the mid and lower regions of the range (e.g. hydration index values of −3, −4, −5, −6, −7, −8 and −9) may indicate dehydration. A sub-range including hydration index values at the middle of the range, including the mid-point of the range and values either side of the mid-point of the range (e.g of −3, −4, −5, and −6) may indicate mild dehydration. A sub-range including hydration index values at the lower end of the range (e.g. of −7, −8 and −9) may indicate moderate dehydration.

A sub-range at the lower end of the range (e.g. including hydration index values of −7, −8 and −9) may correspond to USG values of between 1.020 and 1.030. A sub-range including hydration index values at and either side of the mid-point of the range (e.g. of −3, −4, −5 and −6) may correspond to USG values of approximately 1.020. A sub-range including hydration index values at the upper end of the range (e.g. of 0, −1 and −2) may correspond to USG values of approximately 1.010. A sub-range including hydration index values at the upper end of the range (e.g. of 0, −1 and −2) may correspond to USG values of between 1.010 and 1.020.

A sub-range including hydration index values at the lower end of the range (e.g. of −7, −8, −9) may correspond to UO values of approximately 1100. A sub-range including hydration index values at and either side of the mid-point of the range (e.g. of −3, −4, −5, and −6) may correspond to UO values of between 700 and 850. A hydration index at the upper end point of the range (e.g. of 0) may correspond to UO values of approximately 500. A hydration index having a value towards the upper endpoint but not at the upper endpoint of the range (e.g. of −1) may correspond to UO values of approximately 650. A hydration index of a lower value, e.g., −2, may correspond to UO values of approximately 700.

A sub-range including hydration index values at the lower end including the lower endpoint of the range (e.g. hydration index values of −7, −8, and −9) may correspond to SO values of above 300. A sub-range including hydration index values at and either side of the mid-point of the range (e.g. of −3, −4, −5, −6) may correspond to SO values between 295 and 300. A sub-range including hydration index values at the upper end of the range including the upper endpoint of the range (e.g. of 0, −1 and −2) may correspond to SO values of between 280 and 295. A hydration index at the upper endpoint of the range (e.g. of 0) may correspond to an SO value of approximately 280. A hydration index of e.g. −2 may correspond to an SO values of approximately 295.

A sub-range including hydration index values at the lower end of the range and including the lowest endpoint of the range (e.g. of −7, −8 and −9) may correspond to fluid loss values of approximately between 6% and 9%. A sub-range including hydration index values including the mid-point of the range and values either side of the mid-point, e.g. of −3, −4, −5, and −6 may correspond to fluid loss values of approximately between 2% and 6%. A sub-range including hydration index values at the upper end of the range and including the upper endpoint of the range (e.g. of 0, −1, and −2) may correspond to fluid loss values of approximately between 0 and 2%.

The hydration rank may include a hydration status of the user, wherein the hydration status is a given grade on a hydration status scale.

Each hydration status on the hydration status scale may be a clinical hydration status. In this way, a clinical hydration status of a user may be determined or indicated.

Each hydration status on the hydration status scale may map onto a respective output of a standard clinical point of care test. Each hydration status on the hydration status scale may map onto a respective range of outputs of a standard clinical point of care test.

In this way, a clinically relevant qualitative assessment of hydration status, or a clinical hydration status, may be provided.

Each hydration status on the hydration status scale may map onto respective outputs of a plurality of standard clinical point of care tests. Each hydration status on the hydration status scale may map onto respective ranges of outputs of a plurality of standard clinical point of care tests.

In this way, each hydration status on the hydration status scale may relate to a combination of standard clinical point of care tests. Thus, a more accurate indication of a user clinical hydration status may be provided, because the accuracy of the derived clinical hydration status, as compared to the users actual hydration status, is not limited by the inaccuracies of a single standard point of care test.

A clinical hydration status of overhydration may correspond to UO values of <500 or >80 and <500. A clinical hydration status of normal hydration may correspond to UO values of 500 to 700. A clinical hydration status of dehydration may correspond to UO values of 700 to 1200.

A clinical hydration status of overhydration may correspond to USG values of 1.001 to <1.005. A clinical hydration status of normal hydration may correspond to USG values of 1.005 to <1.020. A clinical hydration status of dehydration may correspond to USG values of 1.020 to 1.040.

A clinical hydration status of overhydration may correspond to SO values of >265 and <285. A clinical hydration status of normal hydration may correspond to SO values of 285 to <295. A clinical hydration status of dehydration may correspond to SO values of 295 to >320.

A clinical hydration status of overhydration may correspond to body mass measurement of weight gain. A clinical hydration status of normal hydration may correspond to a body mass measurement of 0% to <2% weight loss. A clinical hydration status of mild dehydration may correspond to a body mass measurement of 2% to <6% weight loss. A clinical hydration status of moderate dehydration may correspond to a body mass measurement of 6% to <10% weight loss. A clinical hydration status of severe dehydration may correspond to a body mass measurement of >10%.

The hydration rank may include both the hydration index and the hydration status of the user. The model may include an index model which derives the hydration index, and a status model which derives the hydration status.

The sensor may be an optical sensing module (i.e. an optical sensor) on the wearable device.

In this way, an indication of clinical hydration status may be derived using an optical measurement. Further, an indication of the hydration status of the user may be provided non-invasively. An indication of the hydration status of the user may be provided without requiring a sample to be taken from the user.

The optical sensing module may comprise a laser. The optical sensing module may comprise a plurality of lasers. Each laser of the plurality of lasers may operate at a wavelength that is different from the wavelength of the others. The optical sensing module may be configured to drive the plurality of lasers one at a time. The optical sensing module may be configured to operate the plurality of lasers in a cycle according to a pre-determined schedule.

In this way, the optical sensing module may require fewer detectors. The optical sensing module may require only one detector. Advantageously then, the optical sensing module may be cheaper and simpler to manufacture than an optical sensing module which requires more detectors.

In one or more embodiments, the optical sensing module may have a sampling rate of 50,000 samples per second or fewer. In one or more embodiments, the sampling rate may be 1,000 samples per second or more. The data acquired from the optical sensing module may be an average of the samples. A laser on-time for the laser or for each of the plurality of lasers be 200 microseconds or more. As an example, the number of samples acquired in 200 microseconds may be 10. Two samples may be discarded in the processing. An example of a laser off-time for the laser or for each of the plurality of lasers may be 100 microseconds. As an example, the number of samples acquired in 100 microseconds may be 5. As an example, the time to perform 60 cycles may be 20 milliseconds. In one or more embodiments, the number of samples acquired in 20 milliseconds may be 1000 or more. A total measurement time may be 10 seconds or less. As an example, the number of cycles in 10 seconds may be 500. A plurality of total measurements may be taken. There may be an interval of 15 seconds between each total measurement.

The laser or the plurality of lasers may emit light in a wavelength band which is sensitive to changes in water concentration within the interstitial space. The laser or the plurality of lasers may emit light in the wavelength band which covers wavelengths between at least 350 nm and no more than 2500 nm. The laser or the plurality of lasers may emit light in the visible wavelength band. The visible wavelength band may cover wavelengths roughly from 300 nm to 780 nm. The laser or the plurality of lasers may emit light in the infrared wavelength band. The laser or the plurality of lasers may emit light in the near-infrared wavelength band. The near infrared wavelength band may cover wavelengths roughly from 780 nm to 1000 nm. The laser or the plurality of lasers may emit light in the short wavelength infrared wavelength band. The short wavelength infrared wavelength band may cover wavelengths from roughly from 1000 nm to 2500 nm. The laser or a laser within the plurality of lasers may emit light at 970 nm, 1200 nm, 1450 nm, 1950 nm, 2766 nm, 2898 nm, or 6097 nm.

The optical sensing module may comprise one or more optical outputs for light originating from the laser or the plurality of lasers. Light from the laser or the plurality of lasers may exit the optical sensing module via one or more optical output ports. The optical sensing module may comprise a mirror to take the light from the plane of the optical sensing module and translate it into a direction more suitable for interrogating the surface. The direction may be orthogonal or substantially orthogonal to the plane of the optical sensing module.

The optical sensing module may include a transmitter photonic integrated circuit (PIC). The optical sensing module may comprise a substrate. The substrate may be a silicon substrate. The transmitter PIC may be located on the substrate. The transmitter PIC may include the laser or the plurality of lasers. The transmitter photonic integrated circuit (PIC) may be a silicon or silicon nitride photonic integrated circuit.

In use, light emitted from a laser of the optical sensing module may reflect or backscatter from a layer of the skin of the user. The optical sensing module may be configured to receive light backscattered from the skin of the user.

The optical sensing module may comprise a detector. The detector may be a photodetector. The detector may be located on the transmitter PIC such that the PIC is a transmitter/receiver PIC. The detector may be located separately from the transmitter PIC. The photodetector may be a silicon-based photodetector. The photodetector may be an InGaAs-based photodetector. The photodetector may be a germanium photodetector. The photodetector may be located on a receiver PIC that is vertically integrated and mounted on the same substrate as the transmitter PIC. The optical sensing module may comprise a plurality of detectors.

The optical sensing module may comprise an optical manipulation region. The optical manipulation region may comprise one or more of an optical modulator, an optical multiplexer, and additional optical manipulation elements.

The optical sensing module may be that disclosed in WO 2021/116766, the contents of which are incorporated herein by reference in its entirety.

In this way, the optical sensing module may have the ability to continuously take data. Therefore, the computer-implemented method may continuously receive data. Therefore, the hydration status of the user may be continuously monitored. Further, the hydration rank of the user may be provided in real time.

The optical sensing module may comprise LEDs instead of lasers. LEDs may cheaper and simpler to manufacture than lasers. Lasers may allow for a more accurate indication of the hydration status of the user.

The bodily parameter data may be a body tissue absorption spectrum. The absorption may be in the water band. In some examples, the wavelength of the laser or a laser in the plurality of lasers may correspond to the wavelength of a water absorption peak.

In this way, there may be provided a more direct indication of the hydration status of the user, as compared to standard clinical tests which measure proxies for hydration status. Further, a more accurate indication of hydration status of the user may be provided.

The model may include a machine learning model. The model may include a regression model. The model may include a classifier. The model may include a logistic regression model. The model may include a partial least squares (PLS) regression model. The model may include a principal component analysis (PCA) model. The PCA model may be applied before the regression model. The PCA model may be applied before the classifier.

The model may have been trained using training data. The training data may include a plurality of training datasets, each of the training datasets comprising bodily parameter data, and each of the training datasets acquired form the wearable device. The training data may include clinical labels. Each training dataset may be associated with a respective one of the clinical labels. Each of the clinical labels may include an output of a standard clinical point of care test. The clinical data which is used to derive the output of the standard clinical point of care test may have been acquired at the same time as or at a similar time to the acquisition of the corresponding training dataset, for example within the same 5 minute interval, 15 minute interval, or 1 hour interval. Each of the clinical labels may include a plurality of outputs of a respective plurality of standard clinical point of care tests.

The model trained or generated in this way is not limited to a machine learning model.

The model may include an offline model. The offline model may have been trained using batch training data.

The training data may have been acquired from a single subject. The single subject may be the user of the wearable device. The training data may have been acquired from a plurality of subjects.

The model may comprise one or more pre-processing steps. The one or more pre-processing steps may comprise applying a statistical model to the data acquired from the sensor to validate the data acquired from the sensor.

In one or more embodiments, the computer-implemented method may comprise a step of detecting whether the bodily parameter data has been acquired from a human body.

Validating the data may comprise determining whether the data has been acquired from a human user. In addition, or alternatively, validating the data may comprise determining whether the data is anomalous data.

In this way, the effect of any anomalous data, or any incorrectly acquired data on the accuracy of the hydration status indication is reduced.

The statistical model may be generated using a plurality of training datasets, each dataset comprising bodily parameter data. The plurality of training datasets may have been acquired from a plurality of subjects. To validate the data acquired from the sensor, the statistical model may determine whether the data acquired from the sensor falls within a pre-determined number of standard deviations, for example within 2 standard deviations, of the plurality of training datasets. To validate the data acquired form the sensor, the statistical model may calculate a Mahalonobis distance metric between the data acquired form the sensor and the plurality of training datasets, and determine whether the Mahalonobis distance metric is within a given threshold.

The one or more pre-processing steps may comprise applying a baseline correction to the data. Applying the baseline correction to the data may comprise subtracting baseline data from the acquired data. The baseline data may be derived from data previously acquired from the user. The baseline data may be average data acquired from the user. The average data may have been derived from data acquired from the user over a long time period, for example, over 24 hours, 1 week, or 1 month.

In this way, the effect of noise in the data may be reduced. In this way, the indication of the user's hydration status may be more accurate.

The computer-implemented method may comprise acquiring other sensor information.

The other sensor information may be acquired from the sensor on the wearable device. The other sensor information may be acquired from an additional sensor. The additional sensor may be external to the wearable device. The additional sensor may be on the wearable device. The other sensor information may be obtained from other bodily parameter data related to the user. The other bodily parameter data may be an optical spectrum.

The other sensor information may include clinically relevant information. The other sensor information may include one or more of body temperature information obtained from a temperature sensor, heart rate information obtained from a heart rate sensor, blood oxygen saturation information obtained from a blood oxygen saturation sensor, respiratory rate information obtained from a respiratory rate sensor, hydration information obtained from a hydration sensor, accelerometer and motion information obtained from an accelerometer or a motion sensor, heart rate variability information obtained from a heart rate sensor, alcohol concentration, sleep/wake information obtained from a sleep sensor, and blood pressure information obtained from a blood pressure sensor.

The heart rate sensor, the blood oxygen saturation sensor and the respiration rate sensor may be a PPG sensor. The blood pressure sensor and the heart rate sensor may be an SPG sensor. The temperature sensor may be a short-wavelength infrared sensor. The heart rate information and the heart rate variability information may be obtained from an electrocardiogram.

The other sensor information may be or may be measured using physiological indicators. Physiological indicators may include tissue perfusion or ischemia, infection, decompensation, pain, performance, overtraining, movement/activity, core body temperature, resting heart rate, real-time heart rate, maximum heart rate, heart rate thresholds, VO2 or VO2 maximum, intensity, sleep quality, sleep disturbance, apnea hypopnea index (AHI), oxygen desaturation index (ODI), metabolic equivalent of tasks (METs), metabolic health, caloric cost, or general health status.

For example, the blood oxygen saturation information may be an oxygen desaturation index. The heart rate information may be a VO2 measurement or a maximum VO2 measurement. The heart rate information may be an MET measurement. The respiration rate information may be an apnea-hypopnea index or a respiratory disturbance index.

The computer-implemented method may comprise acquiring user input information. The user input information may be acquired from a user input into the wearable device. The user input information may be acquired from a user input into an external device which may be a mobile device such as a mobile phone. The user input information may include one or more of weight information, height information, activity information, diet information, fluid intake information, sodium intake information, illness information, intoxication information and blood pressure information.

The user input information may include a value on a clinically relevant scale. For example, weight information may include a mass in kg, or pounds. Weight information may include a body mass index (BMI). Height information may include a height in cm or inches. Activity information may include an amount of calories burnt. Activity information may include information from which an amount of calories burnt could be calculated, for example a type of exercise and a duration of the exercise. Activity information may also include duration of an activity completed, type of activity completed or other information related to the user's experience of the activity, such as perceived exertion. Diet information may include a food group (such as fat, carbohydrate or protein) consumed. Diet information may include an amount of calories consumed. Diet information may include information from which an amount of calories consumed could be calculated, for example a type of food and an amount of food. Fluid intake information may include a volume of fluid consumed. Fluid intake information may include a type of fluid consumed, for example water or an electrolyte fluid. Illness information may include a temperature. Illness information may include a type of diagnosed illness, a duration of illness or other information related to symptoms. Intoxication information may include a number of alcohol units consumed. Intoxication information may include information from which a number of alcohol units consumed could be calculated, for example a type of alcohol and a volume consumed. Intoxication information may include a number of days in which alcohol has been consumed. Blood pressure information may include a measurement in mmHg. Blood pressure information may include a ratio of systolic pressure to diastolic pressure, where each pressure may be a measurement in mmHg.

The computer-implemented method may comprise acquiring a learnt basal hydration rank of the user or a learnt basal bodily parameter data of the user. Basal bodily parameter data may mean average bodily parameter data over a prolonged period for example over 24 hours, 1 week or 1 month. Basal bodily parameter data may be bodily parameter data acquired when the user is at rest. A basal hydration rank may mean an average hydration rank over a prolonged period for example over 24 hours, 1 week or 1 month. A basal hydration rank may be derived from data acquired when the user is at rest, the data including basal bodily parameter data.

The learnt basal hydration rank or basal bodily parameter data may be acquired from a memory. The memory may be located in the wearable device or in an external device which may be a mobile device such as a mobile phone.

The computer-implemented method may further comprise applying a basal hydration model to the bodily parameter data. The basal hydration model may take the learnt basal bodily parameter data as an input. The basal hydration model may derive whether the bodily parameter data is a pre-determined threshold away from the basal bodily parameter data of the user.

The computer-implemented method may further comprise applying a basal hydration model to the hydration rank. The basal hydration model may take the learnt hydration rank as an input. The basal hydration model may compare the derived hydration rank and the basal hydration rank and, based on this comparison, may determine whether the derived hydration rank is a pre-determined threshold away from a basal hydration rank of the user.

The computer-implemented method may comprise, when the basal hydration model determines that the bodily parameter data is more than the pre-determined threshold away from a user's basal bodily parameter data, alerting the user. The computer-implemented method may comprise, when the basal hydration model determines that the derived hydration rank is more than a pre-determined threshold away from a user's basal hydration rank, alerting the user.

The alert may be output by the wearable device. The alert may be output by an external device. The alert may be a haptic, aural or a visual alert. For example, the alert may be a visual indication on the wearable device that the user is out of their basal hydration range.

In this way, a physical output may be provided to the user when the user is out of their basal hydration range.

The learnt basal bodily parameter data or the learnt basal hydration rank may have been learnt using a machine learning model. The learnt basal bodily parameter data or the learnt basal hydration rank may have been learnt in a calibration period of the computer-implemented method. The calibration period may be an initial period in which a user is using the computer-implemented method. The calibration period may be between 1 day and 21 days. The calibration period may be between 7 days and 14 days. The learnt basal bodily parameter data or the learnt basal hydration rank may have been learnt in a plurality of calibration sub-periods within the calibration period. Each calibration sub-period may be between 1 minute and 1 hour. Each calibration sub-period may be between 5 minutes and 15 minutes. Each of the calibration sub-periods may be a period in which the user has just woken up, for example a period in which the user has woken up within the last 5 minutes, 15 minutes, 30 minutes or 1 hour.

The learnt basal bodily parameter data of the user may have been learnt for changing other sensor information and/or for changing user input information. The learnt basal hydration rank of the user may have been learnt for changing other sensor information and/or for changing user input information.

The training data used to learn the basal hydration rank or the basal bodily parameter data may include a plurality of training basal datasets, the training basal datasets including bodily parameter data, and the training basal datasets acquired from the wearable device. The training data may comprise a plurality of context labels. Each training basal dataset may be associated with a respective one of the context labels. Each of the context labels may include training other sensor information and/or training user input information. The training other sensor information may be acquired at the same time as or at a similar time to the acquisition of the corresponding training basal dataset, for example within the same 5 minute interval, 15 minute interval, or 1 hour interval. The training user input information be acquired at the same time as or at a similar time to the acquisition of the corresponding training basal dataset, for example within the same 5 minute interval, 15 minute interval, or 1 hour interval.

The training user input information may include weight information, height information, activity information, diet information, fluid intake information, illness information, intoxication information, or blood pressure information. The training other sensor information may include body temperature obtained from a temperature sensor, activity information obtained from an accelerometer, heart rate information obtained from a heart rate sensor or blood pressure information obtained from a blood pressure sensor.

The parameters being learnt in this way does not limit the model used to learn the parameters to being a machine learning model.

In this way, basal bodily parameter data or a basal hydration rank of a user may be learnt. Further the basal bodily parameter data or the basal hydration rank may be correlated with user input information and/or other sensor information.

In one or more embodiments, the computer-implemented method may comprise storing a hydration status cause data table, the hydration status cause data table associating causes of a clinical hydration status with stored other sensor information and/or stored user input information respectively; and, optionally, when a hydration rank is derived which indicates that the clinical hydration status of the user is a pre-determined clinical hydration status; comparing acquired other sensor information and/or user input information with stored other sensor information and/or stored user input information respectively and, based on this comparison. The computer-implemented method may further comprise a step of selecting a cause of a clinical hydration status, and, optionally, outputting the selected cause of the clinical hydration status to the user.

The computer-implemented method may comprise, when a hydration index is derived which falls within a pre-determined sub-range, comparing acquired other sensor information and/or user input information with stored other sensor information and/or user input information respectively, and based on this comparison, selecting a cause of a clinical hydration status. When a hydration index is derived which falls within a sub-range other than the pre-determined sub-range, the computer-implemented method may not carry out these comparison and selection steps.

The computer-implemented method may comprise outputting the selected cause of the clinical hydration status to the user. The cause of the clinical hydration status may be output to the user in real time.

The pre-determined clinical hydration status may be dehydration. The pre-determined clinical hydration status may be mild dehydration, moderate dehydration or severe dehydration. The pre-determined clinical hydration status may be overhydration. The pre-determined clinical hydration status may be mild overhydration, moderate overhydration or severe overhydration.

The pre-determined sub-range may correspond to a clinical hydration status of dehydration. The pre-determined sub-range may correspond to a clinical hydration status of mild dehydration, moderate dehydration or severe dehydration. The pre-determined sub-range may correspond to a clinical hydration status of overhydration. The pre-determined sub-range may correspond to a clinical hydration status of mild overhydration, moderate overhydration or severe overhydration.

In this way, clinically relevant factors may be taken into account to derive the cause of a clinical hydration status of the user.

A cause of dehydration may include an active cause of dehydration, a passive cause of dehydration or an illness or condition.

The hydration status cause data table may associate causes of a hydration status with types of hydration status. For example, the hydration status cause data table may associate causes of dehydration with types of dehydration. Types of dehydration may include hyopotonic, hypertonic and isotonic. The computer-implemented method may comprise outputting a type of hydration status to the user, where the type of hydration status is associated with the selected cause of the clinical hydration status.

The hydration status cause data table may associate types of hydration status with stored other sensor information and/or stored user input information.

The computer-implemented method may comprise, when a hydration rank is derived which indicates that the clinical hydration status of the user is a pre-determined clinical hydration status, comparing acquired other sensor information and/or user input information with stored other sensor information and/or user input information respectively, and based on this comparison, selecting a type of a clinical hydration status. When a hydration rank is derived which indicates that the user's clinical hydration status is not the pre-determined clinical hydration status, the computer-implemented method may not carry out these comparison and selection steps.

The computer-implemented method may comprise, when a hydration index is derived which falls within a pre-determined sub-range, comparing acquired other sensor information and/or user input information with stored other sensor information and/or user input information respectively, and based on this comparison, selecting a type of a clinical hydration status. When a hydration index is derived which falls within a sub-range other than the pre-determined sub-range, the computer-implemented method may not carry out these comparison and selection steps.

The computer-implemented method may comprise outputting the selected type of the clinical hydration status to the user. The type of the clinical hydration status may be output to the user in real time.

The computer-implemented method may comprise storing a recommendation data table.

The recommendation data table may associate recommendations with stored hydration ranks. The computer-implemented method may comprise comparing the derived hydration rank with the stored hydration ranks and, based on this comparison, selecting a recommendation to output to the user. The computer-implemented method may further comprise outputting the selected recommendation to the user.

The recommendations may be actions for the user to take, for example “drink water” or “stop consuming water”.

The selected recommendation may be output to the user in real time.

The selected recommendation may be one which is clinically understood to improve the hydration status of the user.

In this way, a recommendation appropriate to improving the clinical hydration status of the user may be output to the user. An improved hydration status of the user may be one which is closer to euvolemia.

The recommendation data table may associate recommendations with stored other sensor information. The recommendation data table may associate recommendations with stored user input information. The computer-implemented method may comprise comparing acquired other sensor information with stored other sensor information. Selecting the recommendation to output to the user may be further based upon this comparison. The computer-implemented method may comprise comparing acquired user input information with stored user input information. Selecting the recommendation to output to the user may be further based upon this comparison.

In this way, the recommendation provided to the user may be more effective at improving the clinical hydration status of the user, by taking into account other clinically relevant information.

If the derived hydration rank indicates that the clinical hydration status of the user is severe dehydration, the selected recommendation may include a prompt to ask for help and/or to seek medical attention. If the derived hydration rank indicates that the clinical hydration status of the user is severe dehydration, the selected recommendation may include a prompt for a user to input user-input information about any other symptoms they may have into a device. The device may be the wearable device or an external device.

If the derived hydration rank indicates that the clinical hydration status of the user is mild overhydration, the selected recommendation may include a prompt to stop consuming water and fluids.

If the derived hydration rank indicates that the clinical hydration status of the user is moderate to severe overhydration, the selected recommendation may include a prompt to ask for help and/or to seek medical attention. If the derived hydration rank indicates that the clinical hydration status of the user is moderate to severe overhydration, the selected recommendation may include a prompt to stop consuming water and fluids. If the derived hydration rank indicates that the clinical hydration status of the user is moderate to severe overhydration, the selected recommendation may include a prompt for a user to input user-input information about any other symptoms they may have.

The computer-implemented method may comprise storing a rehydration fluid type data table. The rehydration fluid type data table may associate types of rehydration fluids with stored other sensor information. The rehydration fluid type data table may associate types of rehydration fluids with stored user input information. Types of rehydration fluid may include, for example, water or an electrolyte fluid.

The computer-implemented method may comprise, when a hydration rank is derived which indicates that the clinical hydration status of the user is dehydration, comparing acquired other sensor information with stored other sensor information and, based on this comparison, selecting a type of rehydration fluid, and outputting the selected type of rehydration fluid to the user.

The computer-implemented method may comprise, when a hydration rank is derived which indicates that the clinical hydration status of the user is dehydration, comparing acquired user input information with stored user input information and, based on this comparison, selecting a type of rehydration fluid, and outputting the selected type of rehydration fluid to the user.

When a hydration rank is derived which indicates that the clinical hydration status of the user is not dehydration, the computer-implemented method may not carry out these comparison and selection steps.

The clinical hydration status being dehydration may include the clinical hydration status being mild dehydration, moderate dehydration or severe dehydration.

If the derived hydration rank indicates that the clinical hydration status of the user is mild dehydration, and if the other sensor information and/or user input information indicates that the user has not undergone physical activity and that the user is not under thermal stress, the recommended fluid type may be water. If the derived hydration rank indicates that the clinical hydration status of the user is mild dehydration, and if the other sensor information and/or user input information indicates that the user has undergone physical activity or that the user is under thermal stress, or is undergoing gastrointestinal problems, the recommended fluid type may be electrolyte fluid and/or water.

Gastrointestinal problems may include acute vomiting and/or diarrhea.

If the derived hydration rank indicates that the clinical hydration status of the user is moderate dehydration, and if the other sensor information and/or user input information indicates that the user has not undergone physical activity and that the user is not under thermal stress, the recommended fluid type may be electrolyte fluid and/or water. If the derived hydration rank indicates that the clinical hydration status of the user is moderate dehydration, and if the other sensor information and/or user input information indicates that the user has undergone physical activity, is undergoing gastrointestinal problems, or that the user is under thermal stress, the recommended fluid type may be electrolyte fluid and/or water.

Whether or not the recommended fluid type is an electrolyte fluid may depend upon a user-input of sodium intake.

The rehydration fluid type data table may associate types of rehydration fluids with stored types of dehydration.

The computer-implemented method may comprise comparing a selected type of dehydration selected from the hydration status cause data table with stored types of dehydration and, based on this comparison, selecting a type of rehydration fluid. The computer implemented method may further comprise outputting the selected type of rehydration fluid to the user.

In this way, a rehydration fluid suitable for rehydrating the user, based on data which may indicate a cause of dehydration of the user, may be output to the user.

The computer-implemented method may comprise storing a rehydration fluid volume data table.

The rehydration fluid volume data table may associate volumes of rehydration fluid with stored hydration ranks.

The computer-implemented method may comprise, when a hydration rank is derived which indicates that the clinical hydration status of the user is dehydration, comparing a derived hydration rank with stored hydration ranks and, based on this comparison, selecting a volume of rehydration fluid, and outputting the selected volume of rehydration fluid to the user.

When a hydration rank is derived which indicates that the clinical hydration status of the user is not dehydration, the computer-implemented method may not carry out these comparison and selection steps.

The rehydration fluid volume data table may associate volumes of rehydration fluid with stored other sensor information. The rehydration fluid volume data table may associate volumes of rehydration fluid with stored user input information. The rehydration fluid volume table may associate volumes of rehydration fluid with other factors such as a type of rehydration fluid.

The computer-implemented method may comprise, when a hydration rank is derived which indicates that the clinical hydration status of the user is dehydration, comparing acquired user input information with stored user input information and, based on this comparison, selecting a volume of rehydration fluid, and outputting the selected volume of rehydration fluid to the user. The computer-implemented method may comprise, when a hydration rank is derived which indicates that the clinical hydration status of the user is dehydration, comparing acquired other sensor information with stored other sensor information and, based on this comparison, selecting a volume of rehydration fluid, and outputting the selected volume of rehydration fluid to the user.

When a hydration rank is derived which indicates that the clinical hydration status of the user is not dehydration, the computer-implemented method may not carry out these comparison and selection steps.

In this way, a volume of rehydration fluid suitable for rehydrating the user, based on a derived hydration rank, which may indicate how dehydrated the user is, may be output to the user. Further, a volume of rehydration fluid suitable for rehydrating the user, based on data which may indicate a cause of dehydration of the user, may be output to the user.

The computer-implemented method may comprise storing a rehydration schedule data table. The rehydration schedule data table may associate a schedule by which rehydration fluid should be consumed with stored hydration ranks. The schedule may include a volume of rehydration fluid. The schedule may include a sub-volume of rehydration fluid and a time at which to drink the sub-volume of the rehydration fluid. The schedule may include a type of rehydration fluid.

The computer-implemented method may comprise, when a hydration rank is derived which indicates that the clinical hydration status of the user is dehydration, comparing a derived hydration rank with stored hydration ranks and, based on this comparison, selecting a schedule by which rehydration fluid should be consumed, and outputting the selected schedule to the user. When a hydration rank is derived which indicates that the clinical hydration status of the user is not dehydration, the computer-implemented method may not carry out these comparison and selection steps.

The rehydration schedule data table may associate a schedule by which rehydration fluid should be consumed with stored other sensor information. The rehydration schedule data table may associate a schedule by which rehydration fluid should be consumed with stored user input information.

The computer-implemented method may comprise, when a hydration rank is derived which indicates that the clinical hydration status of the user is dehydration, comparing acquired other sensor information with stored other sensor information and, based on this comparison, selecting a schedule by which rehydration fluid should be consumed, and outputting the selected schedule to the user. The computer-implemented method may comprise, when a hydration rank is derived which indicates that the clinical hydration status of the user is dehydration, comparing acquired user input information with stored user input information and, based on this comparison, selecting a schedule by which rehydration fluid should be consumed, and outputting the selected schedule to the user. When a hydration rank is derived which indicates that the clinical hydration status of the user is not dehydration, the computer-implemented method may not carry out these comparison and selection steps.

In this way, a rehydration fluid schedule suitable for rehydrating the user, based a hydration rank which may indicate how dehydrated the user is, may be output to the user. Further, a rehydration fluid schedule suitable for rehydrating the user, based on data which may indicate a cause of dehydration of the user or a type of dehydration of the user, may be output to the user.

If the derived hydration rank indicates that the clinical hydration status of the user is mild dehydration, and if the other sensor information and/or user input information indicates that the user has not undergone physical activity and that the user is not under thermal stress, the rehydration schedule may include a prompt to consume water. If the derived hydration rank indicates that the clinical hydration status of the user is mild dehydration, and if the other sensor information and/or user input information indicates that the user has undergone physical activity or that the user is under thermal stress, or is undergoing gastrointestinal problems, the rehydration schedule may include a prompt to consume an electrolyte fluid and one or more subsequent prompts to consume water.

If the derived hydration rank indicates that the clinical hydration status of the user is moderate dehydration, and if the other sensor information and/or user input information indicates that the user has not undergone physical activity and that the user is not under thermal stress and has not undergone gastrointestinal problems, the rehydration schedule may include a prompt to consume an electrolyte fluid and one or more subsequent prompts to consume water. If the derived hydration rank indicates that the clinical hydration status of the user is moderate dehydration, and if the other sensor information and/or user input information indicates that the user has undergone physical activity, is undergoing gastrointestinal problems, or that the user is under thermal stress, the rehydration schedule may include a prompt to consume an electrolyte fluid and one or more subsequent prompts to consume water.

Prompts to consume water may continue until euvolemia is reached.

The computer-implemented method may comprise re-deriving the hydration rank. The computer-implemented method may comprise re-selecting a rehydration schedule based upon the re-derived hydration index or hydration status.

The computer-implemented method may comprise storing a reassessment time data table. The reassessment time data table may associate stored reassessment times with stored hydration ranks.

The computer-implemented method may comprise, when a hydration rank is derived which indicates that the clinical hydration status of the user is dehydration, comparing the derived hydration rank with the stored hydration ranks and, based on this comparison, selecting a reassessment time. The computer-implemented method may comprise, after the reassessment time, re-acquiring data and re-deriving the hydration rank to obtain a reassessment hydration rank.

When a hydration rank is derived which indicates that the clinical hydration status of the user is not dehydration, the computer-implemented method may not carry out these comparison and selection steps.

The computer-implemented method may comprise, when the reassessment hydration index indicates that the user is dehydrated, alerting the user.

The computer-implemented method may comprise, when a hydration rank is derived which indicates that the clinical hydration status of the user is overhydration, comparing the derived hydration rank with the stored hydration ranks and, based on this comparison, selecting a reassessment time, and after the reassessment time, re-acquiring data and re-deriving the hydration rank to obtain a reassessment hydration rank.

When a hydration rank is derived which indicates that the clinical hydration status of the user is not overhydration, the computer-implemented method may not carry out these comparison and selection steps.

The computer-implemented method may comprise, when the reassessment hydration rank indicates that the user is overhydrated, alerting the user.

The alert may be output by the wearable device. The alert may be haptic, aural or visual. The alert may include a recommendation to drink. The alert may include a recommendation not to drink.

The reassessment time data table may associate stored reassessment times with other factors such as a type of rehydration fluid.

The computer-implemented method may comprise comparing a selected type of rehydration fluid selected from the rehydration fluid type data table with stored reassessment times and, based on this comparison, selecting a reassessment time.

The stored reassessment times may be clinically relevant. For example, they may be times which are clinically understood to be long enough for any action taken by the user to have had an impact on their hydration status.

In this way, the clinical hydration status of the user may be re-assessed after a time relevant to the initially derived hydration rank of the user and/or relevant to a recommended type of rehydration fluid. Further, there may be a physical output provided to the user when it is determined that the hydration status has not improved after the reassessment time.

The computer-implemented method may comprise receiving a reassessment time user input. The reassessment time user input may cause the computer-implemented method to re-derive the hydration rank of the user after a pre-determined reassessment time. The reassessment time user input may include a selection of a reassessment time by a user. The reassessment time user input may cause the computer-implemented method to re-derive the hydration rank of the user after a user-selected reassessment time.

The pre-determined reassessment time may be clinically relevant. For example, it may be a time which is clinically understood to be long enough for any action taken by the user to have had an impact on their hydration status.

The computer-implemented method may comprise, after an amount of time indicated by the reassessment time user input, re-acquiring data and re-deriving the hydration rank to obtain a reassessment hydration rank. The computer-implemented method may comprise, when the reassessment hydration rank indicates that the user remains dehydrated or overhydrated, alerting the user.

In this way, there may be a physical output provided to the user when it is determined that the hydration status of a user has not improved after a pre-determined period of time.

The computer-implemented method may comprise storing the hydration rank and storing the time at which the hydration rank is derived. The computer-implemented method may comprise obtaining time-correlated hydration rank information from previously derived stored hydration ranks and their corresponding stored times. The computer-implemented method may comprise outputting the time-correlated hydration rank information to the user such that the user can track how their hydration index varies over time.

In this way, an output may be provided which demonstrates the variation of the clinical hydration status of the user over time.

The computer-implemented method may comprise storing the other sensor information and/or user input information. The computer-implemented method may comprise obtaining time-correlated other sensor information and/or time correlated user input information from previously derived stored other sensor information and/or stored user input information and their corresponding stored times. The computer-implemented method may comprise outputting the time-correlated other sensor information and/or time-correlated user input information to the user such that the user can track how they vary over time.

The computer-implemented method may comprise using the time-correlated hydration rank information and the time-correlated other sensor information and/or user input information to correlate the hydration ranks with the other sensor information and/or user input information.

In this way, an output may be provided which demonstrates the impact of clinical factors on the clinical hydration status of the user.

In a second aspect, one or more embodiments of the invention provide a computer-implemented method for determining a hydration status of a user, the computer-implemented method comprising applying a model to bodily parameter data obtained from a user to obtain hydration information related to the user and deriving, from the hydration information, a hydration rank indicative of a hydration status of the user, wherein the hydration rank is a grade on a hydration rank scale.

The computer-implemented invention may include any one or any combination insofar as they are compatible of the features of the computer-implemented method according to the first aspect of the invention.

In a third aspect, one or more embodiments of the invention provide a computer program which when executed causes one or more processors to perform the method according to the first aspect or the second aspect of the invention.

The one or more processors may be components of the wearable device. The one or more processors may be components of a device external to the wearable device, for example a mobile device such as a mobile phone.

In a fourth aspect, embodiments of the invention provide a method for determining a hydration status of a user, the method comprising providing an optical sensing module on a wearable device worn by a user, providing a processor, and carrying out, by the processor, the computer-implemented method according to the first aspect of the invention, wherein the sensor is the optical sensing module on the wearable device.

In a fifth aspect, one or more embodiments of the invention provide a device comprising a processor, the processor configured to carry out the computer-implemented method according to the first aspect or the second aspect of the invention.

The device may comprise a storage medium storing the computer program according to any one or more embodiments of the present invention.

The device may be a wearable device.

The device may be a mobile device such as a mobile phone or tablet.

In a sixth aspect, one or more embodiments of the invention provide a computer-implemented method for deriving a primary physiological index indicative of a physiological status of a user, the computer-implemented method comprising acquiring, from a sensor on a wearable device worn by a user, data including bodily parameter data related to the user, and applying a model to the bodily parameter data to obtain primary physiological information related to the user, and deriving, from the primary physiological information, a primary physiological index indicative of a physiological status of the user wearing the device, wherein the primary physiological index is a given value on a physiological index scale.

### BRIEF DESCRIPTION OF THE DRAWINGS

These and other features and advantages of the present invention will be appreciated and understood with reference to the specification, claims, and appended drawings wherein:

FIG. 1 depicts an example of hydration information in the form of a hydration rank scale;

FIG. 2 shows a flowchart of example steps of the computer-implemented method;

FIG. 3 is a schematic diagram of an optical sensing module that may be configured to carry out the computer-implemented method;

FIG. 4 shows a flowchart of example steps of the computer-implemented method;

FIG. 5 is a depiction of a further example of steps of a computer-implemented method;

FIG. 6 is an example of an output of the computer-implemented invention in the form of a graphical user interface (GUI), for example on a mobile device;

FIG. 7 shows a further flowchart of example steps of the computer-implemented method;

FIG. 8A depicts a further example of hydration information in the form of a hydration rank scale; and

FIG. 8B depicts a further example of hydration information in the form of a hydration rank scale.

### DETAILED DESCRIPTION

The detailed description set forth below in connection with the appended drawings is intended as a description of exemplary embodiments of a computer-implemented method provided in accordance with the present invention and is not intended to represent the only forms in which the present invention may be constructed or utilized. The description sets forth the features of the present invention in connection with the illustrated embodiments. It is to be understood, however, that the same or equivalent functions and structures may be accomplished by different embodiments that are also intended to be encompassed within the spirit and scope of the invention. As denoted elsewhere herein, like element numbers are intended to indicate like elements or features.

One or more embodiments of the present invention provide a computer-implemented method for determining a hydration status of a user. The computer-implemented method comprises acquiring from a sensor 1101 on a wearable device worn by a user, data including bodily parameter data, for example an optical measurement such as an absorption spectrum related to the user.

The method further comprises applying a model to the bodily parameter data to obtain hydration information related to the user. This information could take the form, for example of information from the spectrum about how hydrated the user is (e.g. from the location/height of the peak). The model derives, from the hydration information, a hydration rank 1 indicative of a hydration status of the user, wherein the hydration rank 1 is a given grade on a hydration rank scale 12.

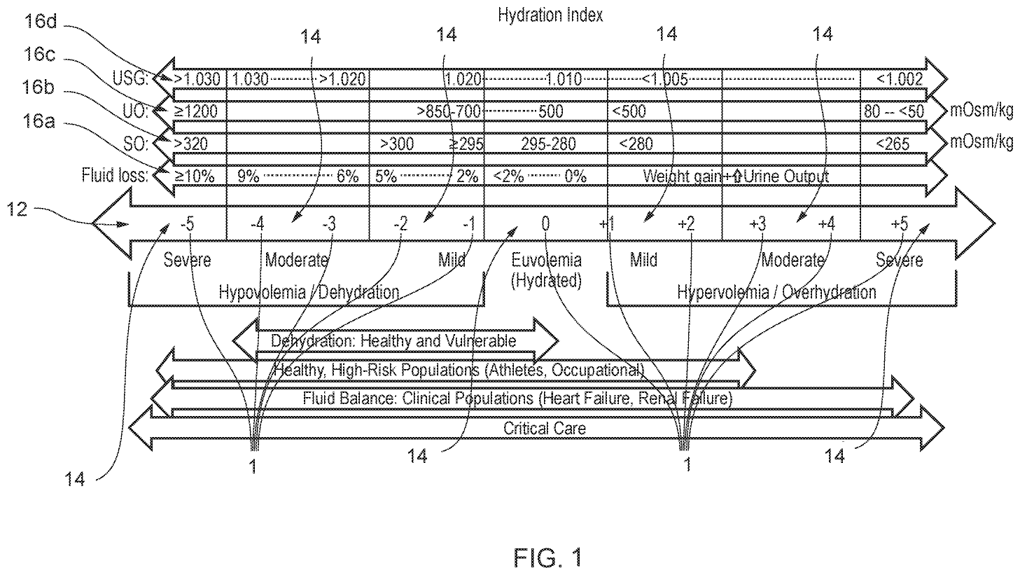

FIG. 1 shows an example of a hydration rank scale 12 according to an embodiment of the present invention. In the embodiment shown in FIG. 1, the hydration rank scale 12 is a scale of hydration indices 10. Each of the hydration indices 1 are integer numbers, and the scale runs from −5 to +5. The model derives a hydration index 1 on this hydration index scale 12.

As shown in FIG. 1, each hydration index 1 on the hydration index scale 12 is indicative of a clinical hydration status of the user. The hydration index 12 scale is sub-divided into a plurality of sub-ranges 14 of hydration index values 1, each of the plurality of sub-ranges 14 corresponding to a different clinical hydration status of the user. These clinical hydration statuses include severe dehydration, moderate dehydration, mild dehydration, euvolemia, mild overhydration, moderate overhydration and severe overhydration. The sub-range including hydration index −5 corresponds to severe dehydration, the sub-range including hydration indices −3 and −4 correspond to moderate dehydration, the sub-range including hydration indices −1 and −2 correspond to mild dehydration, the sub-range including hydration index 0 corresponds to euvolemia, the sub-range including hydration indices +1 and +2 corresponds to mild overhydration, the sub-range including hydration indices +3 and +4 corresponds to moderate overhydration, and the sub-range including hydration index +5 corresponds to severe overhydration.

As further shown in FIG. 1, each hydration index 1 on the hydration index scale 12 maps onto respective ranges of outputs of a plurality of standard clinical point of care tests 16a/b/c/d. In the example shown in FIG. 1, the standard clinical point of care tests 16a/b/c/d are tests of urine osmolality 16c, urine specific gravity 16d, fluid loss 16a and serum osmolality 16b.

Each of these standard clinical point of care tests 16a/b/c/d defines ranges of outputs of the standard clinical point of care test, wherein each of the ranges corresponds to a respective clinical hydration status.

In the example shown in FIG. 1, a hydration index value of −5, which indicates severe dehydration, corresponds to USG values of over 1.030, which also indicates severe dehydration. A sub-range including hydration index values of −3 or −4, which indicate moderate dehydration, corresponds to USG values of between 1.020 and 1.030, which also indicate moderate dehydration. A sub-range including hydration index values of −1 or −2, which indicate mild dehydration, corresponds to USG values of approximately 1.020, which also indicates mild dehydration. A sub-range including hydration index values of −2, −1, 0, 1 or 2, which indicate a hydration status between mild dehydration and mild overhydration, corresponds to USG values of between 1.005 and 1.020, which also indicate a hydration status between mild dehydration and mild overhydration. A hydration index value of 0, which indicates euvolemia corresponds to USG values of approximately 1.010, which also indicates euvolemia. A sub-range including hydration index values of +1 or +2, which indicate mild overhydration, corresponds to a USG value of approximately 1.005, which also indicates mild overhydration. A sub-range including hydration index values of +1, +2, +3, or +4, which indicates a hydration status between mild and moderate overhydration, corresponds to USG values of between 1.002 and 1.005, which also indicates a hydration status between mild and moderate overhydration. A hydration index value of +5, which indicates severe overhydration, corresponds to USG values of less than 1.002, which also indicates severe overhydration.

In other embodiments, the hydration rank scale 12 may be a scale of clinical hydration statuses. In this case, the model derives a clinical hydration status on this clinical hydration status scale. In such embodiments, the clinical hydration statuses (severe dehydration, moderate dehydration, mild dehydration, euvolemia, mild overhydration, moderate overhydration and severe overhydration) shown in FIG. 1, which map onto respective ranges of outputs of the plurality of standard clinical point of care tests, 16a/b/c/d, are directly derived by the model.

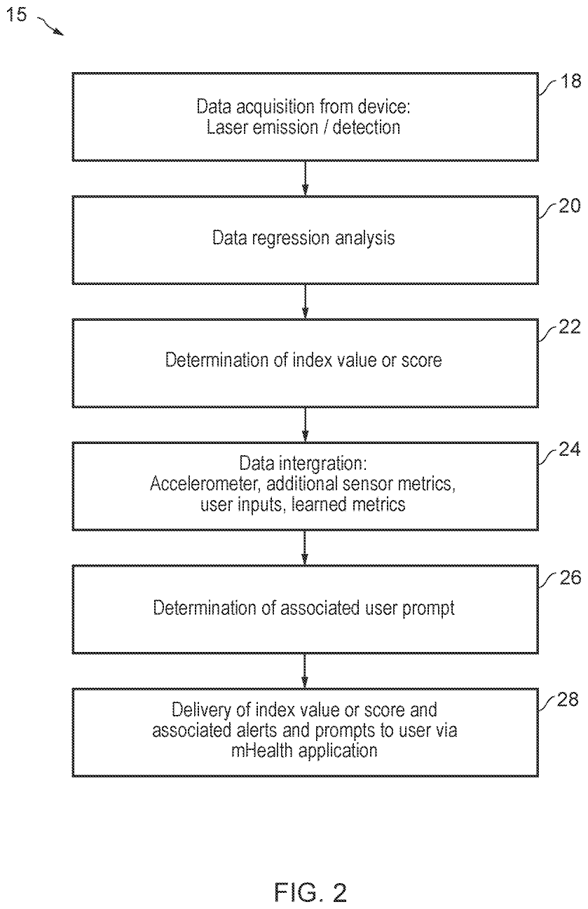

FIG. 2 shows a flow chart 15 setting out steps 18, 20, 22, 24, 26, 28 of the computer-implemented method. Computer-implemented methods according to other embodiments of the present invention may include some, but not all, of the steps shown in FIG. 2. Computer-implemented methods according to other embodiments of the present invention may include additional steps to the steps shown in FIG. 2. The first step 18 of the computer-implemented method shown in FIG. 2 is acquiring data from a sensor on the wearable device.

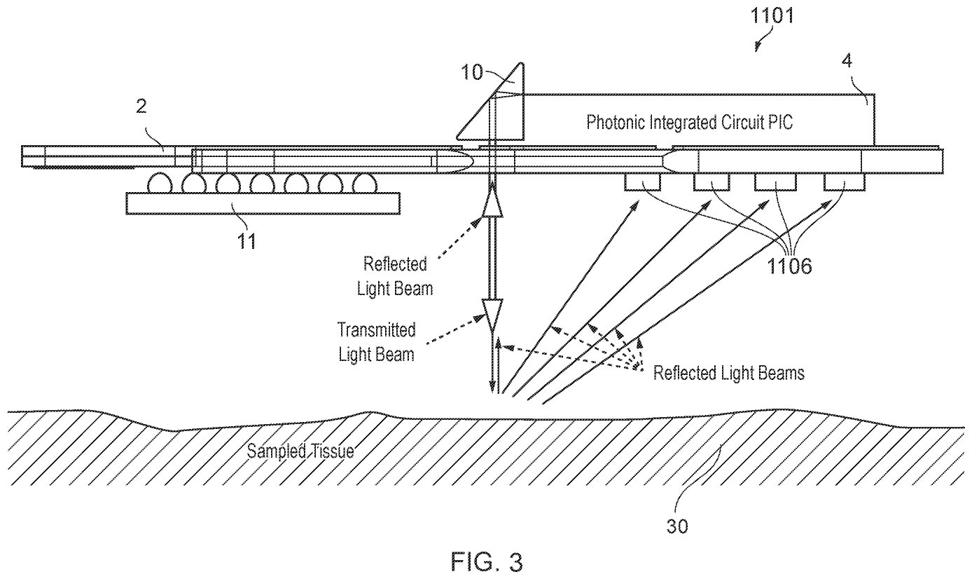

An example of an optical sensing module 1101 will now be described with reference to FIG. 3. The optical sensing module is typically located on the wearable device which acquires the data including the bodily parameter data (e.g. absorption spectrum) related to the user.

The optical sensing module 1101 includes a transmitter photonic integrated circuit (PIC) 4 located on a substrate 2. The PIC 4 includes a plurality of lasers (not visible in FIG. 3), each laser of the plurality of lasers operating at a wavelength that is different from the wavelength of the others. The optical sensing module 1101 is configured to drive the plurality of lasers one at a time. Light from the plurality of lasers exits the PIC 4 and therefore the optical sensing module 101 via one or more optical output ports. A mirror 10 is present to take the light from the plane of the PIC 4 and translate it into a direction more suitable for interrogating the surface. The direction is orthogonal or substantially orthogonal to the plane of the PIC 4.

The plurality of lasers emit light in a wavelength band which is sensitive to changes in water concentration within the interstitial space. The plurality of lasers may emit light in the infrared wavelength band. The plurality of lasers may emit light in the near-infrared wavelength band. The plurality of lasers may emit light in the short wavelength infrared wavelength band. A laser within the plurality of lasers may emit light at 970 nm, 1200 nm, 1450 nm, 1950 nm, 2766 nm, 2898 nm, or 6097 nm, which correspond to water absorption peaks.

In other embodiments, the optical sensing module 1101 may include LEDs in addition to or instead of the lasers.

In use, emitted light from the plurality of lasers is transmitted towards the skin 30 of a user.

Back-scattered light from the surface of the skin 30, and from within a volume below the surface of the skin, returns to the optical sensing module 1101.

A photodetector array comprising photodetector pixels 1106, which collect the backscattered light, forms part of the optical sensing module 1101. In the example shown in FIG. 3, the photodetector array is located on the substrate 2 but is not part of the PIC 4.

An ASIC or microcontroller 11 is located on the substrate 2 of the optical sensing module 1101.

The wearable device carries out the computer-implemented method according to the present invention on a processor (e.g., on a processor of the microcontroller 11 of the wearable device). In other embodiments, an external device such as a mobile phone carries out the computer-implemented method according to the present invention on a processor of the external device.

When the data is acquired from optical sensing module 1110, or from other optical sensing modules, the bodily parameter data is a body tissue absorption spectrum where the absorption is in the water band. The hydration information is a quantitative value associated with the absorption spectrum, for example a wavelength shift of a peak of the absorption spectrum, a height of a peak of the absorption spectrum, or a width of a peak of the absorption spectrum.

In this way, an indication of clinical hydration status of the user can be provided, as the hydration information is sensitive to concentration changes of water within the skin sub-corneal interstitial fluid. As water in the dermis diminishes, the concentration of solutes become higher, thereby changing the degree of water absorption.

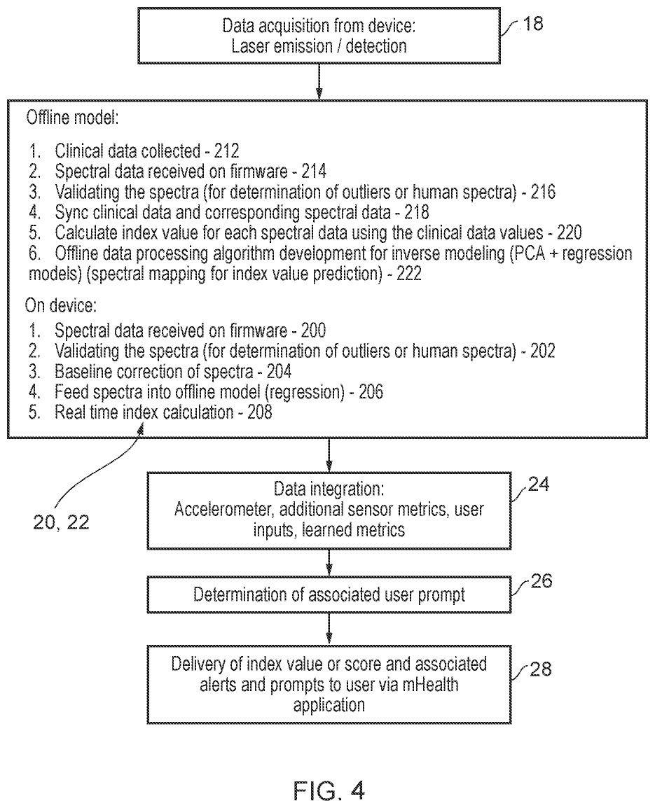

Returning to the flow chart 15 shown in FIG. 2, the second and third steps 20, 22 of the computer-implemented method shown in FIG. 2 respectively include carrying out data regression analysis 20 and determining a hydration index value 22. These steps are shown in more detail in FIG. 4.

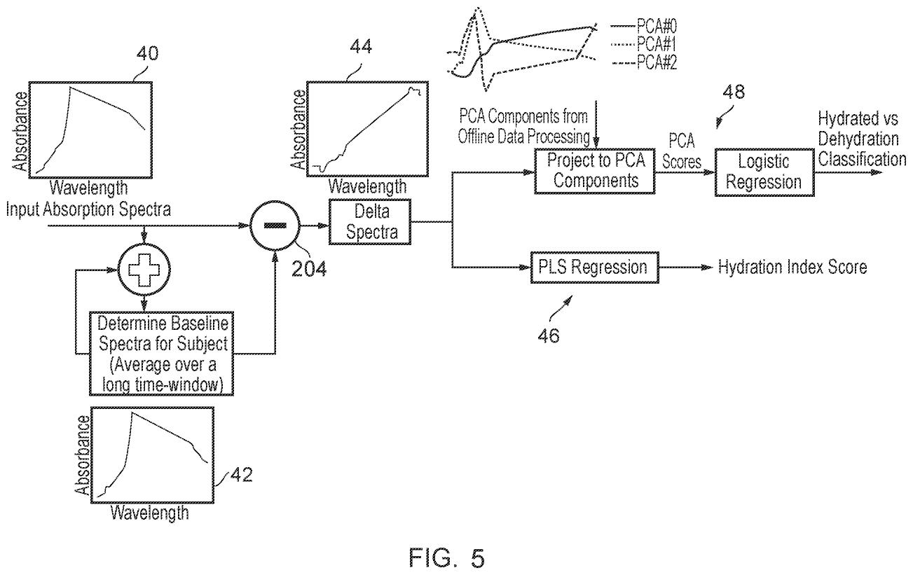

FIG. 4 shows that the second and third steps 20, 22 of the computer-implemented method shown in FIG. 2, include firstly receiving the spectral data 200. Subsequently, pre-processing steps 202, 204 are applied to the data. The first pre-processing step 202 validates the data to determine whether the data has been acquired from a human user, or to determine whether the data includes outlying data. The second pre-processing step 204 applies a baseline correction to the data. This step will be described in more detail below with reference to FIG. 5. Subsequently, the spectra is fed into an offline model 206 and a hydration index value is calculated 208. These steps will be described in more detail below with reference to FIG. 5.

FIG. 5 shows the pre-processing step 204 of applying a baseline correction to the data in more detail. FIG. 5 shows that applying the baseline correction 204 to the data 40 comprises subtracting baseline data 42 from the data 40. The baseline data 42 is the average data acquired from the user over a long time period, for example over 24 hours, 1 week, or 1 month. A delta spectrum 44 results from the baseline correction 42 pre-processing step.

FIG. 5 further shows the step 206 of applying an offline model to the delta spectrum 44 to derive the hydration rank 1. In the example shown in FIG. 5, the offline model derives, as the hydration rank 1, both a hydration index and a hydration status of the user. The offline model includes an index model 46 which derives the hydration index, and a status model 48 which derives the hydration status.

The status model 48 comprises a PCA model and a logistic regression model, where the PCA model is applied to the delta spectrum 44, and the logistic regression model is applied to the output of the PCA model.

The index model 46 is a PLS regression model. The PLS regression model is applied to the delta spectrum.

Further details 210 of how the offline model can generated can be understood with reference to FIG. 4. Each of the index model 46 and the status model 48 of the offline model are generated or trained using training data. The index model and/or the status model may be machine learning models.

A first step 212 outputs of standard clinical point of care tests are received, and in a second step 214 a training dataset is received. A third step 216, is a pre-processing step in which the training dataset is validated to determine whether the training data has been acquired from a human user, or to determine whether the training dataset includes outlying data. In a fourth step 218, the training dataset is mapped to the outputs of the standard clinical point of care tests. In a fifth step 220, a hydration index value is calculated for the training dataset. In a sixth step 222, the offline model is developed.

Thus, training data is collected which comprises a plurality of training datasets, each training dataset being a dataset which comprises bodily parameter information. Each training dataset is associated with a clinical label, where each clinical label is associated with a respective plurality of outputs of standard clinical point of care tests. The clinical data which is used to derive the outputs of the standard clinical point of care tests are acquired at a similar time to the acquisition of the corresponding training dataset, for example within the same 5 minute interval, 15 minute interval or 1 hour interval.

In this way, the offline model effectively maps the data to outputs of standard point of care tests.

Returning to the computer-implemented method 15 shown in FIG. 2, the fourth step 24 of the computer-implemented method is integrating the data with other sensor information, user-input information or learnt metrics.

At this step, 24 the computer-implemented method comprises acquiring other sensor information. The other sensor information may include, for example, body temperature obtained from a temperature sensor, activity information obtained from an accelerometer, heart rate information obtained from a heart rate sensor and blood pressure information obtained from a blood pressure sensor.

User input information may be acquired from a user input into the wearable device or from a user input into an external device such as a mobile phone. The user input information may include, for example, weight information, activity information, diet information, fluid intake information, illness information and intoxication information.

The computer-implemented method may further comprise a step of acquiring a learnt basal hydration rank or basal bodily parameter data of the user. The basal hydration rank or basal bodily parameter data of the user may be learnt in a calibration period of the computer-implemented method using a machine learning model. The training data for the machine learning model may comprise a plurality of training basal datasets comprising bodily parameter data acquired from the wearable device, and a respective plurality of context labels. Each training basal dataset may be associated with a respective one of the context labels. Each of the context labels may include training other sensor information and/or training user input information. The training other sensor information or user input information is acquired at the same time as or at a similar time to the acquisition of the corresponding training basal dataset, for example within the same 5 minute interval, 15 minute interval or 1 hour interval.

Returning to the computer implemented method 15 shown in FIG. 2, the fifth step 26 of the computer-implemented method is determining a user prompt.

The user prompt which is determined may include a cause of a hydration status, a recommendation, a type of rehydration fluid to consume, a volume of rehydration fluid for the user to consume, a rehydration schedule for the user to follow, time-correlated hydration information, an indication that the user's hydration status is outside of a pre-determined basal hydration range, or an indication that the user remains dehydrated or overhydrated after a reassessment time. How each of these user prompts is determined will now be described.

To determine the cause of a hydration status, the computer-implemented method may comprise storing a hydration status cause data table which associates causes of a hydration status with stored other sensor information and/or stored user input information. The computer-implemented method may comprise, when a hydration rank is determined which indicates that the user's clinical hydration status is a pre-determined clinical hydration status, comparing acquired other sensor information and/or user input information with stored other sensor information and/or user input information respectively, and based on this comparison, selecting a cause of a clinical hydration status.

The pre-determined clinical hydration status may be dehydration or overhydration.

For example, the derived hydration index may indicate that the user is dehydrated, and the other sensor information may include temperature information which indicates that the user has a high temperature. The cause of dehydration associated with this temperature information may be, for example, that the user is ill, or that the user is overheated.

To determine a recommendation, the computer-implemented method comprises storing a recommendation data table which associates recommendations with stored hydration ranks. The computer-implemented method comprises comparing the derived hydration rank with the stored hydration ranks respectively and, based on this comparison, selecting a recommendation to output to the user.

For example, the derived hydration rank may indicate that the user is dehydrated. The recommendation associated with this hydration rank may be for the user to drink water.

The recommendation data table may further associate recommendations with stored other sensor information and/or user input information. The computer-implemented method may comprise comparing acquired other sensor information and/or acquired user input information with stored other sensor information and/or stored user input information respectively. Selecting the recommendation to output to the user may be further based upon this comparison.

For example, the derived hydration rank may indicate that the user is dehydrated, and activity information obtained from an accelerometer may indicate that the user is doing/has just finished doing exercise. The recommendation associated with this activity information for this hydration rank may be to rest, or to consume electrolyte fluid.

To determine a type of rehydration fluid to consume, the computer-implemented method comprises storing a rehydration fluid type data table. The rehydration fluid type data table associates types of rehydration fluids with stored other sensor information and/or stored user input information.

The computer-implemented method comprises, when a hydration rank is derived which indicates that the clinical hydration status of the user is dehydration, comparing acquired other sensor information and/or acquired user input information with stored other sensor information and/or stored user input information respectively and, based on this comparison, selecting a type of rehydration fluid.

For example, the derived hydration rank may indicate that the user is dehydrated, and activity information obtained from an accelerometer may indicate that the user is doing/has just finished doing exercise. The rehydration fluid selected in this case may be an electrolyte fluid.

To determine a volume of rehydration fluid for the user to consume, the computer-implemented method comprises storing a rehydration fluid volume data table which associates volumes of rehydration fluid with stored hydration ranks.

The computer-implemented method comprises, when a hydration rank is derived which indicates that the clinical hydration status if the user is dehydration, comparing a derived hydration rank with stored hydration ranks and, based on this comparison, selecting a volume of rehydration fluid.

The rehydration fluid volume data table may further associate volumes of rehydration fluid with stored other sensor information and/or user input information.

The computer-implemented method may comprise, when a hydration rank is derived which indicates that the clinical hydration status of the user is dehydration, comparing acquired other sensor information with stored other sensor information and, based on this comparison, selecting a volume of rehydration fluid. Alternatively, or in addition, the computer-implemented method may comprise, when a hydration rank is derived which indicates that the clinical hydration status of the user is dehydration, comparing acquired user input information with stored user input information and, based on this comparison, selecting a volume of rehydration fluid.

To determine a rehydration schedule for the user to follow, the computer-implemented method may comprise storing a rehydration schedule data table which associates a schedule by which rehydration fluid should be consumed with stored hydration ranks.

The computer-implemented method may comprise, when a hydration rank is derived which indicates that the clinical hydration status of the user is dehydration, comparing a derived hydration rank with stored hydration ranks and based on this comparison, selecting a schedule by which rehydration fluid should be consumed.

The rehydration fluid volume data table may further associate a schedule by which rehydration fluid should be consumed with stored user input information and/or other sensor information.

The computer-implemented method may comprise, when a hydration rank is derived which indicates that the clinical hydration status of the user is dehydration, comparing acquired other sensor information or user input information with stored other sensor information or user input information respectively and, based on this comparison, selecting a schedule by which rehydration fluid should be consumed.

For example, the hydration rank may indicate that the user is moderately dehydrated and temperature information acquired from a temperature sensor may indicate that the user is overheated. The selected schedule by which rehydration fluid should be consumed may be selected based on these factors. The schedule may include a type of rehydration fluid to consume, an overall volume of rehydration fluid to consume, sub-volumes of the overall volume of rehydration fluid to consume and times at which the sub-volumes of rehydration fluid should be consumed.

To determine time-correlated hydration information, the computer-implemented method may comprise a step of storing the hydration rank and storing the time at which the hydration rank is derived. The computer-implemented method thus comprises obtaining time-correlated hydration rank information from previously derived stored hydration ranks and their corresponding stored times.

The computer-implemented method may further comprise a step of storing the other sensor information and/or user input information. The computer-implemented method may thus comprise obtaining time-correlated other sensor information and/or time correlated user input information from previously derived stored other sensor information and/or user input information and their corresponding stored times.

To determine whether the user's hydration status is outside of a pre-determined basal hydration range, the computer-implemented method may comprise a step of applying a basal hydration model to the bodily parameter data, or the derived hydration rank.

In one or more embodiments, the basal hydration model takes the learnt basal bodily parameter data or learnt hydration rank as an input. The basal hydration model may derive whether the derived bodily parameter data, or derived hydration rank is more than a pre-determined threshold away from a user's basal bodily parameter data or basal hydration rank respectively.

To determine whether the user remains dehydrated or overhydrated after a reassessment time, the computer-implemented method comprises re-deriving the hydration rank after a reassessment time. The reassessment time may be a pre-determined reassessment time, a user-input reassessment time, or a reassessment time based upon the initially derived hydration rank.

Returning to the method 15 shown in FIG. 2, the sixth step 28 of the computer-implemented method shown in FIG. 2 is delivery of an output to the user.

The output may be displayed to the user on the wearable device, or on an external device such as a mobile phone.

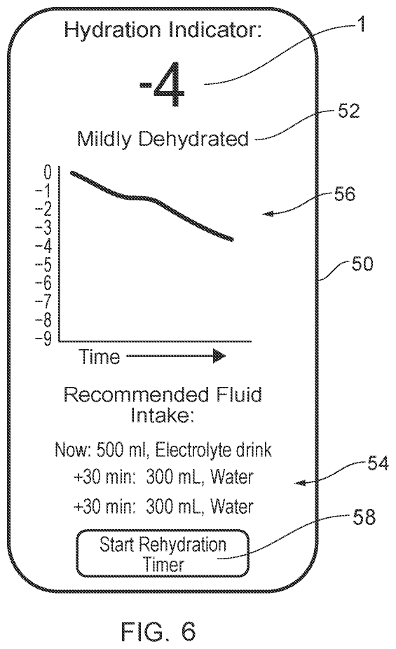

FIG. 6 shows an example of an output being displayed to a user on a mobile device which is a mobile phone 50. The output includes a derived hydration index 1 of −4, and a derived clinical hydration status 52 of mildly dehydrated.

The output further includes a recommended rehydration schedule 54 which is to drink 500 ml of electrolyte fluid at a current time, 300 ml of water after half an hour, and another 300 ml of water after an hour.

The output also includes time-correlated hydration information 56 which shows that the user's hydration index has fallen from 0 to −4 over a period of time.

FIG. 6 also shows that a user input may be received on a portion 58 of the user interface of the mobile device which displays “Start Rehydration Timer” to the user to cause the computer-implemented method to re-derive the hydration status or the hydration index of the user after a pre-determined reassessment time. The computer-implemented method may comprise, when a hydration status or a hydration index, derived after the reassessment time, indicates that the user remains dehydrated after the reassessment time, outputting an alert to the user.

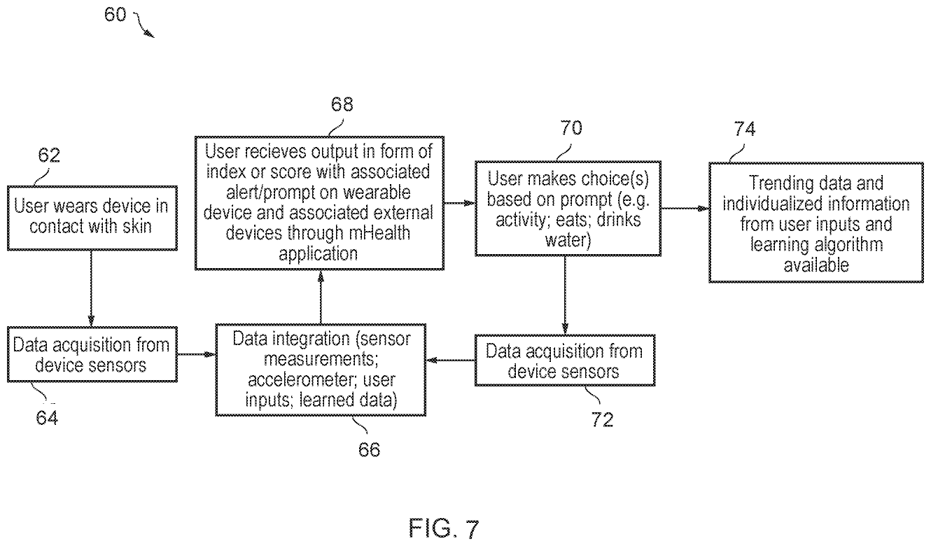

FIG. 7 shows a flow chart 60 for a method which includes embodiments of the computer-implemented method of the present invention. At a first step 62, the user wears a wearable device such that it is in contact with their skin. At a second step 64, data is acquired from sensors of the wearable device. At a third step 66, other sensor information, user input data, and learnt parameters are acquired. At a fourth step 68, an output is provided to the user. At a fifth step 70, the user makes a choice based on the output. At a sixth step 72, data is re-acquired from the sensors. The third step 66 to the sixth step 72 are carried out in a loop. At a seventh step 74, which follows the fifth step, time-correlated information and learnt parameters are acquired.

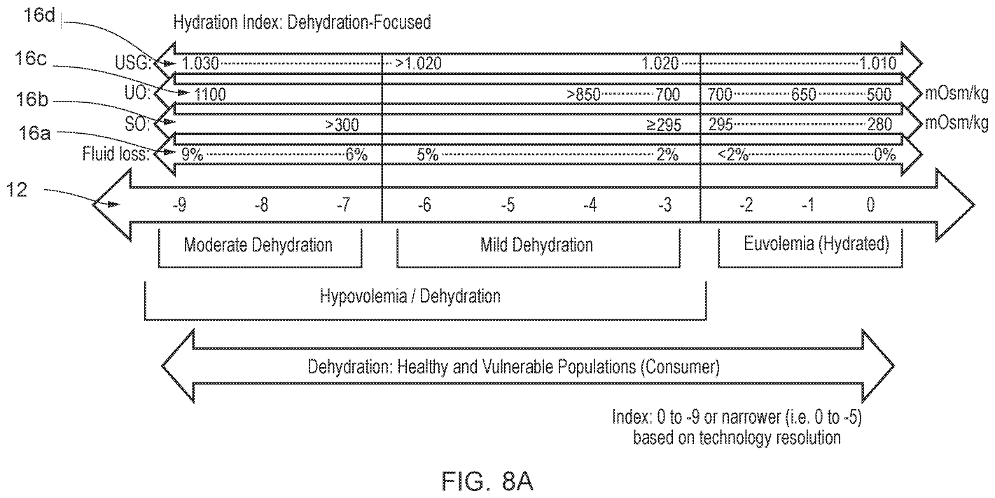

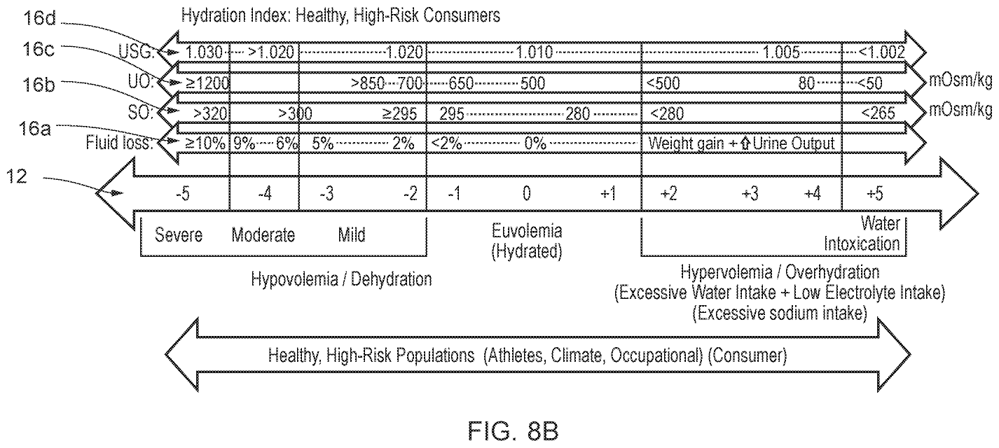

FIGS. 8A and 8B show examples of different hydration index scales 12 to that shown in FIG. 1. As shown in FIGS. 1, 8A and 8B, the hydration index scale 12, and how its hydration indices 1 map onto output of standard clinical point of care tests 16a/b/c/d, may depend on the use case for the computer-implemented method. For example, FIG. 1 shows a hydration index scale 12 running from −5 to +5 which may be used for critical care patients. FIG. 8A shows a hydration index scale 12 running from −9 to 0 which may be used for both healthy and vulnerable users. FIG. 8B shows a hydration index scale 12 running from −5 to +5 which may be used for healthy, high-risk users such as athletes. Populations that may benefit from the present invention include elderly populations, endurance athletes, those travelling to altitude or hot climates, and those in occupations with a high risk of dehydration and overheating.

Although exemplary embodiments of a computer-implemented method have been specifically described and illustrated herein, many modifications and variations will be apparent to those skilled in the art. Accordingly, it is to be understood that a computer-implemented method constructed according to principles of this invention may be embodied other than as specifically described herein. The invention is also defined in the following claims, and equivalents thereof.

1. A computer-implemented method for determining a hydration status of a user, the computer-implemented method comprising:
acquiring, from a sensor on a wearable device worn by a user, data including bodily parameter data related to the user; and
applying a model to the bodily parameter data to obtain hydration information related to the user; wherein
the model derives, from the hydration information, a hydration rank indicative of a hydration status of the user, wherein the hydration rank is a given grade on a hydration rank scale.

2. The computer-implemented method of claim 1 wherein each hydration rank on a hydration ranks scale maps onto a respective output of a standard clinical point of care test.

3. The computer-implemented method of claim 2 wherein the standard clinical point of care test is a test of urine osmolality, urine specific gravity, fluid gain, fluid loss, increases or decreases in body weight or mass representing fluid gain or fluid loss, respectively, or serum osmolality.

4. The computer-implemented method of claim 1 wherein the hydration rank is a hydration index, and wherein the hydration index is a given value on a hydration index scale.

5. The computer-implemented method of claim 4 wherein each hydration index on the hydration index scale maps onto a respective output of a standard clinical point of care test.

6. The computer-implemented method of claim 4 wherein the hydration index scale is sub-divided into a plurality of sub-ranges of hydration index values, each of the plurality of sub-ranges corresponding to a different clinical hydration status of the user, and wherein the method further comprises:
determining which sub-range of the plurality of sub-ranges the hydration index value falls within.

7. The computer-implemented method of claim 1 wherein the hydration rank is a clinical hydration status of the user.

8. The computer-implemented method of claim 6 further comprising:
outputting the clinical hydration status of the user.

9. The computer-implemented method of claim 4 further comprising:
outputting the hydration index.

10. The computer-implemented method of claim 1 wherein the sensor is an optical sensing module.

11. The computer-implemented method of claim 10 wherein the optical sensing module comprises a laser.

12. The computer-implemented method of claim 11 wherein the optical sensing module comprises a plurality of lasers, each laser of the plurality of lasers operating at a wavelength that is different from the wavelength of the others.

13. The computer-implemented method of claim 12 wherein the optical sensing module is configured to operate each laser one at a time.

14. The computer-implemented method of claim 13 wherein the optical sensing module is configured to operate the plurality of lasers in a cycle according to a pre-determined schedule.

15. The computer-implemented method of claim 10 wherein the bodily parameter data is a body tissue absorption spectrum.

16. The computer-implemented method of claim 1 wherein the model includes a regression model.

17. The computer-implemented method of claim 1, further comprising applying a statistical model to the data acquired from the sensor to validate the data acquired from the sensor.

18. The computer-implemented method of claim 1 further comprising:
acquiring other sensor information in addition to the hydration information; and/or,
acquiring user input information.

19. The computer-implemented method of claim 18 wherein the other sensor information includes one or more of body temperature information obtained from a temperature sensor, activity information obtained from an accelerometer, heart rate information obtained from a heart rate sensor and blood pressure information obtained from a blood pressure sensor.

20. The computer-implemented method of claim 18 wherein the user input information includes one or more of weight information, activity information, diet information, fluid intake information, illness information and intoxication information.

21. The computer-implemented method of claim 18, further comprising:
storing a hydration status cause data table, the hydration status cause data table associating causes of a clinical hydration status with stored other sensor information and/or stored user input information respectively; and
when a hydration rank is derived which indicates that the clinical hydration status of the user is a pre-determined clinical hydration status;
comparing acquired other sensor information and/or user input information with stored other sensor information and/or stored user input information respectively and, based on this comparison;
selecting a cause of a clinical hydration status, and
outputting the selected cause of the clinical hydration status to the user.

22. A computer-implemented method for determining a hydration status of a user, the computer-implemented method comprising:
applying a model to bodily parameter data obtained from a user to obtain hydration information related to the user; and
deriving, from the hydration information, a hydration rank indicative of a hydration status of the user, wherein the hydration rank is a given grade on a hydration rank scale.

23. A computer program which when executed causes one or more processors to perform the method of claim 1.

24. A method for determining a hydration status of a user, the method comprising:
providing an optical sensing module on a wearable device worn by a user;
providing a processor; and
carrying out, by the processor, the computer-implemented method of claim 1, wherein the sensor is the optical sensing module on the wearable device.

25. A device comprising a processor, the processor configured to carry out the computer-implemented method of claim 1.

26. The device of claim 25 wherein the device is a wearable device.
